(19) United States
(12) Patent Application Publication
(10) Pub. No.: US 2012/0035103 A1
(43) Pub. Date: Feb. 9, 2012

(54) METHOD FOR ADMINISTRATION OF INSULIN AND PHARMACEUTICAL COMPOSITION THEREOF

(76) Inventors: Dennis J. Pillion, Hoover, AL (US); Elias Meezan, Birmingham, AL (US)

(21) Appl. No.: 13/016,784

(22) Filed: Jan. 28, 2011

Related U.S. Application Data
(60) Provisional application No. 61/299,867, filed on Jan. 29, 2010.

(51) Int. Cl. A61K 38/28; A61P 3/10

(52) U.S. Cl. 514/6.3; 514/5.9

(57) ABSTRACT

The present invention provides pharmaceutical compositions including alkylglycosides admixed with at least one of fast-acting and long-acting insulin analogs for treatment of disorders, such as diabetes.

### CROSS REFERENCE TO RELATED APPLICATION(S)

This application claims the benefit of priority under 35 U.S.C. §119(e) of U.S. Ser. No. 61/299,867, filed Jan. 29, 2010, the entire content of which is incorporated herein by reference.

### BACKGROUND OF THE INVENTION

1. Field of the Invention

The invention relates generally to pharmaceutical compositions and more specifically to administration of pharmaceutical compositions including alkylglycosides and insulin for the treatment of diabetes.

2. Background Information

Patients with diabetes mellitus require precise and timely administration of insulin to maintain normal glycemic control. Continuous subcutaneous insulin infusion (CSII) pumps provide a steady flow of insulin, but require the purchase of expensive equipment and vigilant patient oversight. CSII is further complicated by the short and sometimes variable length of time a device will remain patent after insertion. Occlusion of CSII lines then creates a hazardous situation in which patients are not receiving adequate insulin delivery. Subcutaneous injections of insulin remain the most widely used approach for insulin delivery. Fast-acting and long-acting analogs of insulin have allowed many patients to achieve improved glycemic control, with fast-acting insulin injections before each meal to cover food intake and one or possibly two injections of long-acting insulin to cover insulin requirements between meals and overnight.

The currently available fast-acting insulins (lispro insulin, aspart insulin and glulisine insulin) have distinctly quicker onsets of action compared to regular insulins used previously. Unfortunately, many patients still encounter great difficulty in successfully utilizing injections of these insulins to maintain a glycemic status near normal. Additionally, many Type 2 diabetic patients are averse to beginning insulin therapy because of a reluctance or fear of performing subcutaneous injections. The fact that the fast-acting and long-acting insulin analogs currently available cannot be mixed, and injected together, mandates more injections per day, and this creates a still higher barrier for patients with Type 1 or Type 2 diabetes to overcome.

To circumvent the problems associated with subcutaneous injections or CSII, alternate routes of insulin administration have been explored, but to date these have been found to present barriers to insulin absorption of varying intensities. The pulmonary route of delivery can be utilized without the aid of any absorption-enhancing agent, albeit with a modest bioavailability of approximately 10% of the insulin administered. An inhaled insulin product was developed, tested and shown to be effective. Unfortunately, issues with patient acceptance and pulmonary health, variability of insulin absorption among smokers, and a requirement for monitoring pulmonary function, all contributed to the decision to withdraw this product. While this was certainly a disappointing episode, it provided the proof-of-concept that, with improved technology and refined formulations, an insulin product could be administered by a non-invasive route. This realization is further evidenced by the fact that another inhaled insulin product is in development. Other routes of insulin delivery under consideration include oral, buccal, transdermal and nasal delivery.

The nasal route of delivery, like all alternate routes, offers some potential advantages and disadvantages. The primary challenge to any nasal insulin product is to achieve substantial and reproducible bioavailability from the nasal cavity, a system that is naturally designed to warm and moisten air, and to remove particulate matter, not designed to absorb drugs. Despite the nearly absolute impermeability of the nasal passage to insulin under basal conditions, there is a remarkable increase in insulin absorption when a very small concentration of an alkylglycoside surfactant agent, with the appropriate size and hydrophilic/hydrophobic balance, is added to the formulation. Alkylglycoside surfactants that consist of an alkyl chain 12-14 carbons in length, and a disaccharide, such as dodecyl-β-d-maltoside (DDM), tetradecyl-β-d-maltoside (TDM) or dodecylsucrose, are known to produce a transient increase in the nasal permeability to a wide variety of both peptide and non-peptide drugs. It is important to note that alkylglycosides with side chains shorter than 10 carbons in length, or those linked to a monosaccharide, fail to have this effect on the nasal cavity. Studies with cultured epithelial cells and with intact animals provide evidence that the alkylglycosides increase both paracellular and transcellular insulin absorption.

The effects of alkylglycosides on the permeability of the nasal cavity in vivo are transient, with maximal effectiveness observed immediately after application and gradual reversal over the next 2-4 hours. From a clinical perspective, this pattern of transient loosening of the permeability barrier of the nose overlaps with the most likely pattern of use for a nasal fast-acting insulin product, for instance the nasal insulin would be taken just before meals and produce an immediate hypoglycemic response. Animal experiments have shown that the pharmacokinetic profiles of regular insulin and the fast-acting analog lispro insulin were similar when applied nasally in the presence of TDM, with very rapid absorption of insulin (Tₘₐₓ=15 minutes) and return to baseline insulin values within 1-2 hours. The long-acting insulin analog, glargine insulin, gave different results when applied nasally in the presence of alkylglycosides. Glargine insulin showed a slower onset (Tₘₐₓ=60 minutes) and an extended duration of absorption and action.

However, it remains uncertain whether a fast-acting insulin analog may be mixed with a long-acting insulin analog and be formulated in the presence of an alkylglycoside. Additionally, a mixed insulin formulation that is absorbed into the circulation to produce a blended pharmacokinetic/pharmacodynamic profile would be beneficial in treating a patient with diabetes mellitus.

### SUMMARY OF THE INVENTION

The present invention relates to formulations of alkylglycosides admixed with both one or more of fast-acting and long-acting insulin analogs for treatment of disorders, such as diabetes.

Accordingly, the present invention provides a pharmaceutical composition including at least one insulin analog and an absorption increasing amount of an alkylglycoside. In one embodiment, the pharmaceutical composition includes both a long-acting insulin analog in combination with a fast-acting insulin analog. In some embodiments, the long-acting insulin analog is glargine or detemir insulin and the fast-acting insulin-analog is lispro, aspart, or glulisine. In various embodiments, the alkylglycoside has an alkyl chain including between 10 to 16 carbons, such as dodecyl-β-d-maltoside, tridecyl-β-d-maltoside, tetradecyl-β-d-maltoside, sucrose mono-dodecanoate, sucrose mono-tridecanoate, and sucrose mono-tetradecanoate.

In another aspect, the present invention provides a method of introducing one or more insulin analogs into the circulatory system of a subject. The method includes administering the one or more insulin analogs admixed with an alkylglycoside, thereby introducing the insulin analog to the circulatory system of the subject. In various embodiments, the one or more insulin analogs includes a combination of both a long-acting insulin analog in combination with a fast-acting insulin analog. In some embodiments, the long-acting insulin analog is glargine or detemir insulin and the fast-acting insulin-analog is lispro, aspart, or glulisine. In various embodiments, the alkylglycoside has an alkyl chain including between 10 to 16 carbons, such as dodecyl-β-d-maltoside, tridecyl-β-d-maltoside, tetradecyl-β-d-maltoside, sucrose mono-dodecanoate, sucrose mono-tridecanoate, and sucrose mono-tetradecanoate. In various embodiments, the one or more insulin analogs is administered via the oral, buccal, nasal, nasolacrimal, inhalation, pulmonary, transdermal or CSF delivery route.

In another aspect, the present invention provides a method of treating diabetes mellitus in a subject. The method includes administering to a subject a pharmaceutical composition of the present invention.

### BRIEF DESCRIPTION OF THE DRAWINGS

FIG. 1 is a series of graphical representations of plasma insulin levels in rats.

FIG. 1A is a plot of plasma insulin levels in rats administered various fast-acting insulins with or without 0.125% TDM. FIG. 1B is a plot of plasma insulin levels in rats administered various long-acting or regular insulins with or without 0.125% TDM.

FIG. 2 is a series of graphical representations of blood glucose levels in rats. FIG. 1A is a plot of blood glucose levels in rats administered various fast-acting insulins with or without 0.125% TDM. FIG. 1B is a plot of blood glucose levels in rats administered various long-acting or regular insulins with or without 0.125% TDM.

FIG. 3 is a graphical representation of a plot of plasma insulin levels in rats that received various insulins 15 minutes after administration of either 0% or 0.125% TDM.

FIG. 4 is a graphical representation of a plot of blood glucose levels in rats that received various insulins 15 minutes after administration of either 0% or 0.125% TDM.

FIG. 5 is a graphical representation of a plot of plasma insulin levels in rats that received mixed insulin formulations with 0.125% TDM.

FIG. 6 is a graphical representation of a plot of blood glucose levels in rats that received mixed insulin formulations with 0.125% TDM.

### DETAILED DESCRIPTION OF THE INVENTION

The present invention is based on pharmaceutical compositions that include mixtures of insulins and alkylglycosides that provide exemplary pharmacokinetic and pharmacodynamic profiles of the insulins in treatment of diseases, such as diabetes mellitus. The present invention is based, in part, on observations resulting from administering mixtures of fast-acting and long-acting insulins to anesthetized, hyperglycemic rats in the presence and absence of alkylglycosides. The fast-acting analogs, aspart insulin, lispro insulin and glulisine insulin, were all rapidly absorbed from the nose when applied individually with alkylglycoside. As discussed in the Example and shown in the Figures, one long-acting insulin analog, glargine insulin, was also surprisingly absorbed from the nose when applied individually in the presence of alkylglycoside. However, the other long-acting insulin analog, detemir insulin, was not soluble when formulated with alkylglycoside. A series of mixtures of the three rapid-acting insulins and long-acting glargine insulin were formulated with alkylglycoside and applied nasally. The pharmacokinetic and pharmacodynamic profiles of the insulin mixtures reflected the additive contributions of both the rapid-acting and the long-acting insulins and provided compositions that are expected to match the needs of patients with diabetes mellitus better than those currently available.

Before the present composition and method are described, it is to be understood that this invention is not limited to the particular composition, method, and experimental condition described. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In one embodiment the present invention provides a pharmaceutical composition including one or more insulin analogs and an absorption increasing amount of an alkylglycoside. For example, in one embodiment, the pharmaceutical composition includes at least one fast-acting insulin analog and at least one long-acting insulin analog. One in the art would understand that any fast-acting insulin analog may used with the invention. Fast-acting insulin analogs include, for example, lispro, aspart, and glulisine insulins. Similarly, one in the art would understand that various long-acting insulin analogs may used with the invention, so long as they may be absorbed. Examples of long-acting insulin analogs include glargine and detemir insulin. In exemplary embodiments, glargine is formulated with one or more of lispro, aspart, and glulisine insulins.

As used herein, “alkylglycoside” refers to any sugar joined by a linkage to any hydrophobic alkyl, as is known in the art. Preferably the alkylglycoside is nonionic as well as nontoxic. Alkylglycosides are available from a number of commercial sources and may be natural or synthesized by known procedures, such as chemically or enzymatically.

In various aspects, alkylglycosides of the present invention may include, but not limited to: alkylglycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers.

As described above, the hydrophobic alkyl can thus be chosen of any desired size, depending on the hydrophobicity desired and the hydrophilicity of the saccharide moiety. For example, one preferred range of alkyl chains is from about 10 to about 24 carbon atoms. An even more preferred range is from about 10 to about 16 or about 14 carbon atoms. Similarly, some preferred glycosides include maltose, sucrose, and glucose linked by glycosidic linkage to an alkyl chain of 9, 10, 12, 13, 14, 16, 18, 20, 22, or 24 carbon atoms, for example, nonyl-, decyl-, dodecyl-, tridecyl, and tetradecyl sucroside, glucoside, maltoside, and the like. These compositions are nontoxic, since they are degraded to an alcohol or fatty acid and an oligosaccharide, and amphipathic. Additionally, the linkage between the hydrophobic alkyl group and the hydrophilic saccharide can include, among other possibilities, a glycosidic, thioglycosidic, amide, ureide, or ester linkage.

In sugar chemistry, an anomer is either of a pair of cyclic stereoisomers (designated α or β) of a sugar or glycoside, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, dodecyl β-D-maltoside and dodecyl α-D-maltoside are two cyclic forms of dodecyl maltoside and are anomers. The two different anomers are two distinct chemical structures, and thus have different physical and chemical properties. In one embodiment of the invention, the alkylglycoside for use with the present invention is a β anomer. In an exemplary aspect, the alkylglycoside is a β anomer of dodecyl maltoside, tridecyl maltoside or tetradecyl maltoside.

In one embodiment of the present invention, the alkylglycoside used is a substantially pure alkylglycoside. As used herein a “substantially pure” alkylglycoside refers to one anomeric form of the alkylglycoside (either the α or β anomeric forms) with less than about 2% of the other anomeric form, preferably less than about 1.5% of the other anomeric form, and more preferably less than about 1% of the other anomeric form. In one aspect, a substantially pure alkylgycoside contains greater than 98% of either the α or β anomer. In another aspect, a substantially pure alkylgycoside contains greater than 99% of either the α or β anomer. In another aspect, a substantially pure alkylgycoside contains greater than 99.5% of either the α or β anomer. In another aspect, a substantially pure alkylgycoside contains greater than 99.9% of either the α or β anomer.

The pharmaceutical compositions described herein are formulated for various routes of administration, such as, oral, buccal, nasal, nasolacrimal, inhalation, pulmonary, transdermal or CSF administration. As such, in addition to insulin analogs and alkylglycosides, the compositions may further include one or more of an aggregation inhibitory agent; a charge-modifying agent; a pH control agent; a degradative enzyme inhibitory agent; a mucolytic or mucus clearing agent; a ciliostatic agent; or a membrane penetration-enhancing agent.

Examples of membrane penetration-enhancing agents include cyclodextrins, such as methyl-beta-cyclodextrin; alkylglycosides, such as dodecylmaltoside and tetradecylmaltoside; an aggregation inhibitory agent; a charge-modifying agent; a pH control agent; a degradative enzyme inhibitory agent; a mucolytic or mucus clearing agent; a ciliostatic agent; a membrane penetration-enhancing agent selected from: (i) a cyclodextrin such as methyl-beta-cyclodextrin; an alkylglycoside or other surfactant; (ii) a bile salt; (ii) a phospholipid additive, mixed micelle, liposome, or carrier; (iii) an alcohol; (iv) an enamine; (v) an NO donor compound; (vi) a long-chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); a modulatory agent of epithelial junction physiology; a vasodilator agent; a selective transport-enhancing agent; and a stabilizing delivery vehicle, carrier, mucoadhesive, support or complex-forming species with which the compound is effectively combined, associated, contained, encapsulated or bound resulting in stabilization of the compound for enhanced nasal mucosal delivery, wherein the formulation of the compound with the intranasal delivery-enhancing agents provides for increased bioavailability of the compound in a blood plasma of a subject.

Examples of preservatives that may be used in the compositions of the present invention, include, but are not limited to preservatives such as ethylene diamine tetraacetic acid (EDTA), sodium azide, p-hydroxybenzoate and its analogs, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, chlorobutanol, m-cresol and alkyglycosides such as dodecyl maltoside.

In various embodiments, the compositions described herein may further include one or more excipients including stabilizers, surfactants, antimicrobial agents, osmolarity adjusting agents such as mannitol, sorbitol or sodium chloride.

Further, the compositions described herein may be buffered to have a pH of about 4 to 8, 4.5 to 7.5, 4.5 to 6.5, or 5 to 6. For example, in one embodiment the pharmaceutical compositions of the present invention are buffered such that upon nasal or pulmonary administration of the composition the pH of the nasal or pulmonary mucosa is relatively unperturbed. In various aspects, the pH of the nasal or pulmonary mucosa after administration remains unchanged or maintained within 1 pH point or less of the pH before administration of the pharmaceutical composition. Accordingly, the pH of the nasal or pulmonary mucosa after administration is maintained within less than 1, 0.9, 0.8, 0.7, 0.6, 0.5, 0.4, 0.3, 0.2 or 0.1 pH points of the pH before administration of the pharmaceutical composition.

In various embodiments, the insulin analogs are present in a ratio to allow for an optimal pharmacokinetic profile. For example, the composition can be formulated such that the fast-acting to long-acting insulin ratio is about 20:1, 15:1, 10:1, 9:1, 8:1, 7:1, 6:1, 5:1, 4:1, 3:1, 2:1 1:1, or vice versa.

The present invention also provides a method of introducing one or more insulin analogs into the circulatory system of a subject. The method includes administering the one or more insulin analogs admixed with an alkylglycoside, thereby introducing the insulin analog to the circulatory system of the subject.

The present invention further provides a method of treating diabetes mellitus in a subject. The method includes administering to a subject a pharmaceutical composition of the present invention including one or more insulin analogs admixed with an alkylglycoside, thereby treating diabetes mellitus in the subject.

As described herein, the compositions of the invention may be formulated for, and delivered via any suitable administration route, including, for example, oral, buccal, nasal, nasolacrimal, inhalation, pulmonary, transdermal or CSF delivery routes. The terms “administration” or “administering” as used herein are defined to include the act of providing a pharmaceutical composition of the invention to a subject in need of treatment. While the compositions described herein may be suitable for administration via any well known route, an exemplary administration route is nasal, intranasal or pulmonary administration. As used herein, the terms “nasal”, “intranasal” and “pulmonary” administration are intended to include administration to mucosal tissue lining the nasal cavity and the epithelial linings of the airway (e.g., trachea, bronchus, bronchioles and the like). Accordingly, in an exemplary aspect, the pharmaceutical compositions of the present invention, are formulated into pharmaceutically acceptable forms suitable for nasal and pulmonary administration, such as drops, sprays and inhalants.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, and the like, and primates (including monkeys, chimpanzees, orangutans and gorillas) are included within the definition of subject.

Intranasal administration of the pharmaceutical compositions of the present invention may be achieved by using any one of a number of commercially available metered nasal spray pumps. Manufacturers of such pumps include Pfeiffer and Valois. Dispensing volumes achievable by using these pumps range from approximately 50 μL up to 150 μL. Volumes beyond 150 μL frequently result in the drug running out of the patient's nostrils and unless a mucoadhesive or thickening agent is included in the formulation, volumes in excess of 150 μl, are typically not used.

The dose of insulin analogs, can be modulated by changing either the volume of spray admitted into the nostril or by changing the concentration of a particular insulin analog in the composition. The total amount of insulin analogs to be administered in practicing a method of the invention can be administered to a subject as a single dose or application (e.g., a single nasal application) over a relatively short period of time, or can be administered using a fractionated treatment protocol, in which multiple doses or applications are administered over a prolonged period of time. One skilled in the art would know that the amount of the analog used to treat a subject depends on many factors such as the ailment or disease being treated, the age and general health of the subject as well as the number of treatments to be administered. In view of these factors, the skilled artisan would adjust the particular dose as necessary. In general, the dosage and frequency of administration are determined, initially, using Phase I and Phase II clinical trials. A suitable daily dose of an analog is generally that amount of the analog which is the lowest dose effective to produce a therapeutic effect. Such an effective dose will generally depend upon the factors described above.

If desired, the effective daily dose of the therapeutic peptide may be administered as two, three, four, five, six or more sub-doses administered separately at appropriate intervals throughout the day, optionally, in unit dosage forms (e.g., single nasal applications). There may be a period of no administration followed by another regimen of administration.

It will be understood, however, that the specific dose level and frequency of dosage for any particular patient may be varied and will depend upon a variety of factors including the activity of the analog employed, the metabolic stability and length of action of that compound, the age, body weight, general health, sex, diet, mode and time of administration, rate of excretion, drug combination, the severity of the particular condition, and the host undergoing therapy.

The following examples are intended to illustrate but not limit the invention.

### Example 1

### Nasal Administration of Mixed Insulin Formulations Including Alkylglycosides

This study was designed to address: 1) whether a fast-acting insulin analog could be mixed with a long-acting insulin analog and be formulated in the presence of an alkyglycoside; and 2) whether the mixture could be applied nasally and absorbed into the circulation to produce a blended pharmacokinetic/pharmacodynamic profile that would be suitable for treating a patient with diabetes mellitus.

The following experimental protocols and materials were utilized.

Animals: Studies were performed in Sprague-Dawley male rats (200-450 g) obtained from Charles River Laboratories (Charlotte, N.C.). Standard laboratory food and tap water were available ad libitum. The animal study was conducted according to the principles outlined in the “Guide for the Care and Use of Laboratory Animals,” Institute of Laboratory Animal Resources, National Research Council.

Materials: Glargine insulin (Lantus®) and glulisine insulin (Apidra®) were obtained from Sanofi/Aventis (Bridgewater, N.J.). Detemir insulin (Levemir®), aspart insulin (Novolog®), NPHinsulin (Novolin 125 N®), and regular insulin (Novolin R®) were all obtained from Novo Nordisk Pharmaceuticals, Inc. (Princeton, N.J.). Lispro insulin (Humalog®) was obtained from Eli. Lilly Corp. (Indianapolis, Ind.). Tetradecyl-β-d-maltoside (TDM) was purchased from Anattrace, Inc. (Maumee, Ohio). Xylazine HCl (100 mg/ml) was obtained from Vedco Inc. (St. Joseph, Mo.). Ketamine HCl (100 mg/ml) was obtained from Lloyd Laboratories (Shenandoah, Iowa). Isoflurane was obtained from Minrad Inc. (Bethlehem, Pa.). Heparin (1000 U/ml) was obtained from Elinks-Sin Inc. (Cherry Hill, N.J.). A human insulin specific radioimmunoassay kit was obtained from Millipore Corp. (St. Louis, Mo.).

Analytic Procedures: Rats were anesthetized with 4% isoflurane, initially at a flow rate of 3-4 L/min, and then anesthesia was maintained, when necessary, with 1-2% isoflurane at a flow rate of 1-2 L/min. Rats were then removed from isoflurane and injected with ketamine (100 mg/kg) and xylazine (10 mg/kg) intramuscularly to induce hyperglycemia. Anesthesia was maintained with additional ketamine/xylazine as needed throughout the experiment. Sixty minutes after the first administration of ketamine/xylazine, blood was collected from the tail for the determination of basal glucose and insulin levels. Nose drops (0.02 ml) containing 1 Unit of insulin were administered to the right nares of anesthetized rats in the supine position using a pipettor with a disposable plastic tip. Rats then received a second 0.02 ml dose of the nasal formulation containing 1 Unit of insulin applied to the right nares 2 minutes later. The rats were turned over to the prone position 2 minutes later. This experimental protocol has been utilized to prevent airway obstruction that can occur if nose drops are applied to both nares at the same time and to eliminate leakage of the formulation from the nose.

Nose drops were formulated by mixing one volume of insulin (U-100) with one volume of either 0.9% NaCl or with 0.25% TDM in 0.9% NaCl. In some experiments, phosphate buffered saline was used in place of 0.9% NaCl. No differences were observed in the experiments utilizing phosphate buffered saline. Glucose levels were measured in drops of blood taken from the tip of the rat tail using a glucose meter (Glucometer Elite™, Bayer Corp., Elkhart, Ind.) at various times after the administration of insulin. The upper limit of the glucose meter was 600 mg/dL. Insulin levels were measured using a commercially available human insulin RIA kit. Plasma samples were prepared by collecting rat blood from the tip of the tail in tubes containing 5 Units of heparin.

Statistical Analysis: In nasal drug delivery studies, the maximal concentration (Cₘₐₓ) of insulin and the time tomaximal concentration (Tₘₐₓ) were determined directly from the pharmacokinetic profile. The area under the curve (AUC₀₋₁₈₀) was determined via the linear trapezoidal rule. Results are presented as mean±standard error. Statistical significance was determined with Student's t-test (SigmaStat Software, SPSS, Inc., Chicago, Ill.). Differences with a p value of less than 0.05 were considered significant.

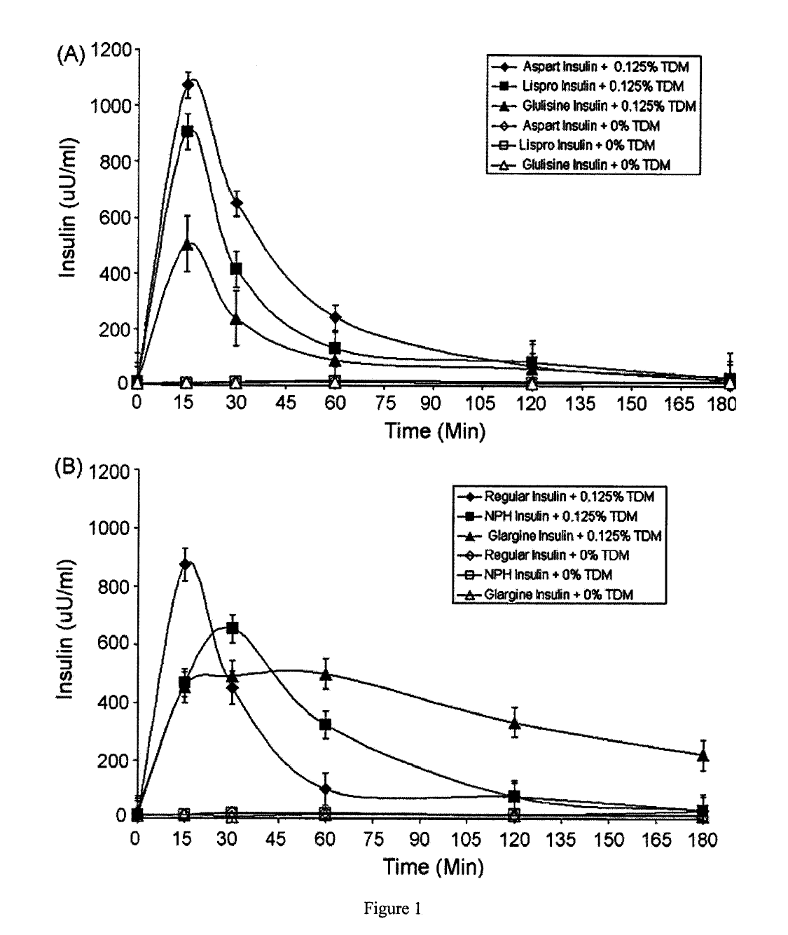

The following results were observed. Previous studies have shown that rats anesthetized with ketamine/xylazine become hyperglycemic and hypoinsulinemic within 60 minutes. This experimental system allowed an exquisitely sensitive platform in which to study the absorption of exogenous human insulin delivered nasally. In this study, seven different forms of insulin were compared. Nasal administration of three different fast-acting insulin analogs, in the presence of 0.125% TDM, caused a rapid and significant increase in plasma insulin levels (FIG. 1A).

FIG. 1 shows plasma insulin levels in rats that received 2 Units of various fast-acting insulins (A) or regular or long-acting insulins (B) with or without 0.125% TDM. Each data point represents the mean±SEM of 3-6 animals.

Maximal insulin levels were obtained within 15 minutes for all three insulin analogs and insulin levels returned to baseline within 120 minutes. Two of the three fast-acting insulin analogs, lispro and aspart, were absorbed more effectively (Cₘₐₓ=907±65_U/ml and 1073±45_U/ml, respectively) than glulisine (Cₘₐₓ=505±99_U/ml). None of the three fast-acting insulin analogs were absorbed when formulated without TDM. Three other forms of insulin (regular, NPH and glargine insulins) were also absorbed from the nasal cavity when formulated with 0.125% TDM (FIG. 1B). Like the fast-acting insulins shown in FIG. 1A, none of these three forms of insulin were absorbed in the absence of TDM. The Tₘₐₓ for NPH insulin (30 minutes) and glargine insulin (60 minutes) were different than the Tₘₐₓ for regular insulin and the fast-acting insulin analogs (15 minutes). Glargine insulin applied nasally in the presence of 0.125% TDM caused a much greater increase in plasma insulin levels 180 minutes after administration than the other forms of insulin tested.

The other long-acting form of insulin, detemir insulin, formed a cloudy mixture when formulated with 0.125% TDM. When this cloudy mixture was applied to rats nasally, under the same conditions as those described above for the other six insulin products, no measurable absorption of insulin was detected. The data in FIG. 1 have been used to determine the total amount of insulin absorbed from the nasal cavity (AUC₀₋₁₈₀) for each of the insulin analogs shown in Table 1 below.

TABLE 1
Effect of tetradecyl-β-d-maltoside (TDM) on the nasal absorption
of various insulins.

| Conditions | | | | |
|---|---|---|---|---|
| 0.125% TDM | | Plasma insulin | | |
| (+/−) | Type of insulin | AUC₀₋₁₈₀ (μU/ml min) | Cₘₐₓ (μU/ml) | Tₘₐₓ (min) |
| (−) | Regular insulin^{a} | N/D | N/D | N/D |
| (+) | Regular insulin | 33,398 ± 2500^{b} | 874 ± 56^{b} | 15 |
| (+) | Lispro insulin | 41,820 ± 3300^{b} | 907 ± 62^{b} | 15 |
| (+) | Aspart insulin | 55,238 ± 4600^{b,c} | 1073 ± 45^{b,c} | 15 |
| (+) | Glulisine insulin | 25,335 ± 1200^{b,c} | 505 ± 99^{b,c} | 15 |
| (+) | NPH insulin | 41,790 ± 3900^{b} | 653 ± 48^{b,c} | 30 |
| (+) | Glargine insulin | 67,170 ± 4800^{b,c} | 500 ± 52^{b,c} | 60 |

Rats received nose drops containing 2 Units of insulin with or without 0.125% TDM.
Plasma insulin levels were determined at various times up to 180 minutes after nasal insulin administration. The AUC values were measured by the trapezoidal rule and compared to the AUC values observed with regular insulin. Data represent mean ± SEM (n = 3-6).
N/D = not detectable.
^{a}Similar results were obtained when other insulins were tested in the presence of 0% TDM.
^{b}Significantly different than the same insulin formulated with 0% TDM (p < 0.05).
^{c}Significantly different than regular insulin formulated with 0.125% TDM (p < 0.05).

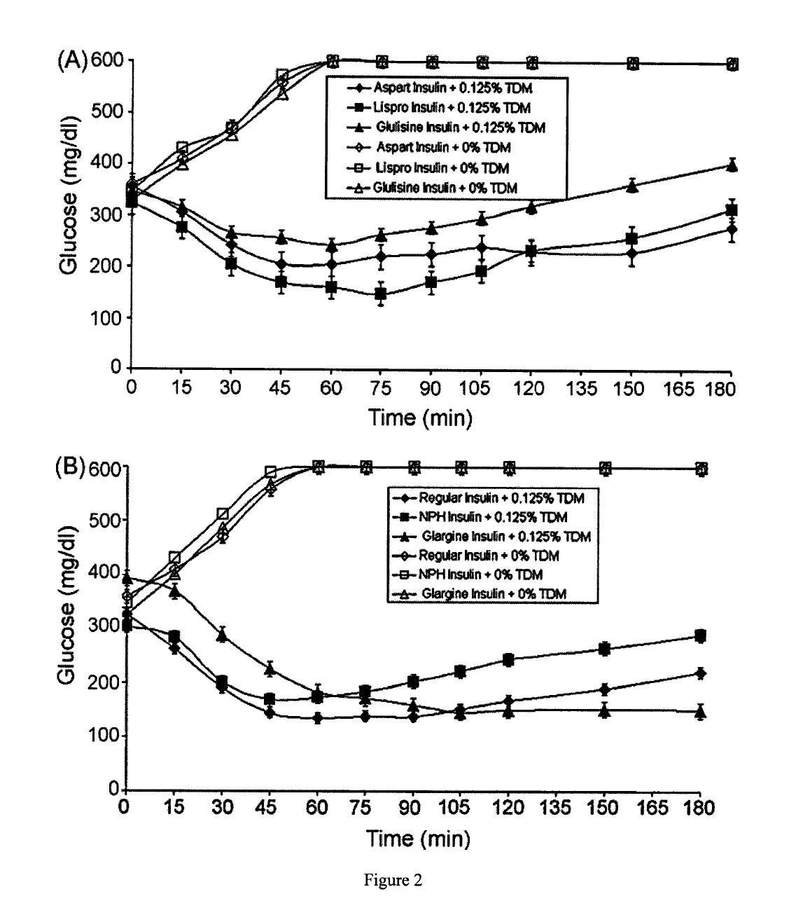

It should be noted that glargine insulin levels had not returned to baseline values 180 minutes after nasal administration. Hence, the values reported for glargine insulin absorption in Table 1 under-represent total glargine insulin absorption when compared to each of the other forms of insulin tested. The total absorption of glulisine insulin (AUC0-180) and the maximal concentration of glulisine insulin (Cₘₐₓ) were consistently less than those observed for the other insulin analogs. Nasal delivery of aspart insulin generated the highest concentration of insulin (Cₘₐₓ) immediately following administration, while nasal delivery of glargine insulin produced the greatest bioavailability of insulin (AUC₀₋₁₈₀) (Table 1). Blood glucose levels were also measured in parallel in the experiments described above to determine the bioavailability of the various insulin analogs, to determine the time course of insulin action following nasal delivery, and to confirm that intact biologically active insulin, rather than a partially degraded but immunologically competent fragment was delivered to the circulation. All three of the fast-acting insulin analogs, formulated with 0.125% TDM and applied nasally, produced a rapid and substantial decrease in blood glucose concentrations compared to the same formulations lacking 0.125% TDM (FIG. 2A). Of the three fast-acting insulin analogs formulated with 0.125% TDM, glulisine insulin produced the smallest hypoglycemic response, consistent with the insulin absorption data presented in FIG. 1A and Table 1.

The effects of all three fast-acting insulin analogs on the glycemic levels of the rats diminished after 180 minutes, again consistent with insulin absorption data presented in FIG. 1A. In FIG. 2B, the effects of regular insulin, NPH insulin, and glargine insulin nose drops formulated with and without 0.125% TDM are presented.

FIG. 2 shows blood glucose levels in rats that received 2 Units of various fast-acting insulins (A) or regular or long-acting insulins (B) with or without 0.125% TDM. Each data point represents the mean±SEM of 3-6 animals.

Unlike the other five forms of insulin tested, long-acting glargine insulin produced a reduction in blood glucose concentration that was still robust after 180 minutes. Hence, the total hypoglycemic response to glargine insulin is under-represented when the AUC0-180 data are compared to the other insulins. Additional experiments are required to define the duration of this effect. When the total hypoglycemic responses to the six forms of insulin were compared directly, glulisine insulin produced the smallest response when applied nasally, whereas regular insulin and glargine insulin produced the largest responses as shown in Table 2 below.

TABLE 2
Effect of TDM on the hypoglyemic response to various insulins.

| | | Blood glucose | |
|---|---|---|---|
| Conditions | | | Blood glucose at 60 |
| 0.125% | | | minutes after insulin |
| TDM | | AUC₀₋₁₈₀ | administration |
| (+/−) | Type of insulin | (mg/dL × min) | (mg/dL) |
| (−) | Regular insulin^{a} | 93,728 ± 2600 | 600 ± 10^{b} |
| (+) | Regular insulin | 32,745 ± 2400^{c} | 135 ± 22^{c} |
| (+) | Lispro insulin | 39,863 ± 3200^{c} | 160 ± 27^{c} |
| (+) | Aspart insulin | 43,470 ± 4300^{c,d} | 205 ± 13^{c,d} |
| (+) | Glulisine insulin | 55,335 ± 4000^{c,d} | 242 ± 15^{c,d} |
| (+) | NPH insulin | 41,730 ± 3100^{c,d} | 174 ± 19^{c} |
| (+) | Glargine insulin | 36,345 ± 3600^{c} | 183 ± 26^{c} |

Rats received nose drops containing 2 Units of insulin with or without 0.125% TDM. The blood glucose levels were measured at various times up to 180 minutes after nasal insulin administration. The AUC values were measured by the trapezoidal rule and compared to the AUC values observed with regular insulin. Basal glucose levels ranged from 303 mg/dL to 393 mg/dL. Data represent mean ± SEM (n = 3-6.)
^{a}Similar results were obtained when other insulins were tested in the presence of 0% TDM.
^{b}Upper limit of glucometer = 600 mg/dL
^{c}Significantly different than the same insulin formulated with 0% TDM (p < 0.05).
^{d}Significantly different than regular insulin formulated with 0.125% TDM (p < 0.05).

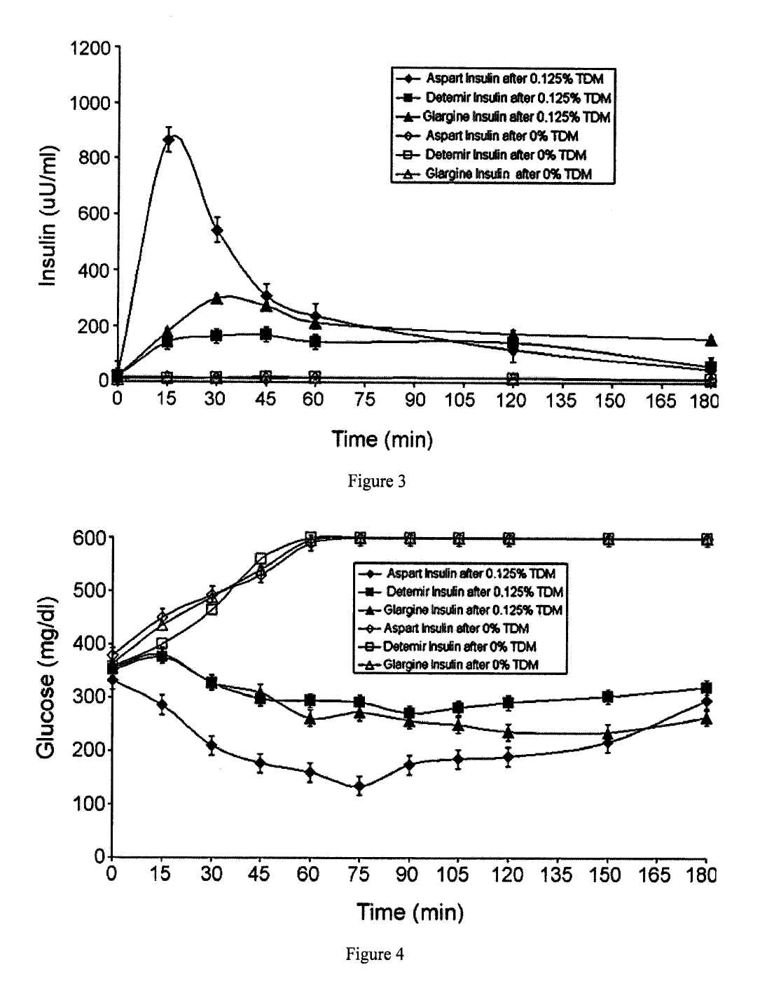

As described above, detemir insulin formed a cloudy mixture when formulated with 0.125% TDM. In order to test the impact of on the nasal absorption of detemir insulin, a different experimental protocol was required. In these experiments, the nasal passages were pre-treated with or without 0.125% TDM. Nose drops containing 2 Units of detemir insulin formulated in 0.9% NaCl were administered 15 minutes later. For comparison, two other forms of insulin, fast-acting aspart insulin and long-acting glargine insulin, were run in parallel. Under these experimental conditions, all three forms of insulin were absorbed from the nasal cavity when applied 15 minutes after 0.125% TDM, but not when applied after 0.9% NaCl (FIG. 3).

Figure three shows plasma insulin levels in rats that received 2 Units of various insulins 15 min after the administration of 0% or 0.125% TDM. Each data point represents the mean±SEM of 3-6 animals.

However, nasal delivery of aspart insulin produced a robust increase in plasma insulin concentrations, whereas glargine insulin and detemir insulin produced progressively smaller responses. Plasma insulin concentrations remained elevated for a longer duration when glargine insulin was applied nasally 15 minutes after 0.125% TDM. Blood glucose concentrations were also obtained in these experiments to determine the time course of insulin action following nasal delivery and to confirm that a biologically active form of insulin had been delivered to the circulation (FIG. 4).

FIG. 4 shows blood glucose levels in rats that received 2 Units of various insulins 15 min after the administration of 0% or 0.125% TDM. Each data point represents the mean±SEM of 3-6 animals.

Fast-acting aspart insulin produced a rapid and more substantial decrease in blood glucose when applied 15 minutes after 0.125% TDM than the long-acting insulin analogs, detemir and glargine. All of these changes in blood glucose concentrations were consistent with the insulin absorption data in FIG. 4.

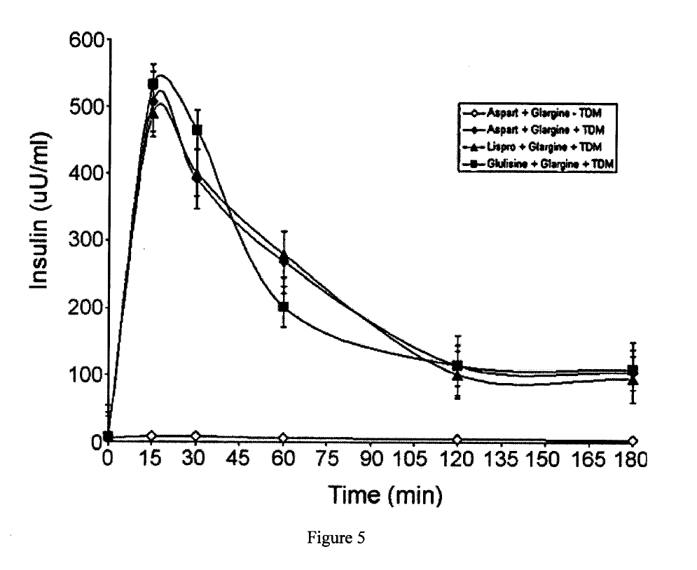

To determine if mixtures of fast-acting and long-acting insulins could be prepared with TDM and used successfully in nasal insulin delivery studies, long-acting glargine insulin (1 Unit) was mixed with either aspart insulin, lispro insulin or glulisine insulin (1 Unit) and applied to rats nasally in the presence or absence of 0.125% TDM (FIG. 5).

FIG. 5 shows plasma insulin levels in rats that received 2 Units of a mixed insulin formulation with 0.125% TDM. Each data point represents the mean±SEM of 3-6 animals.

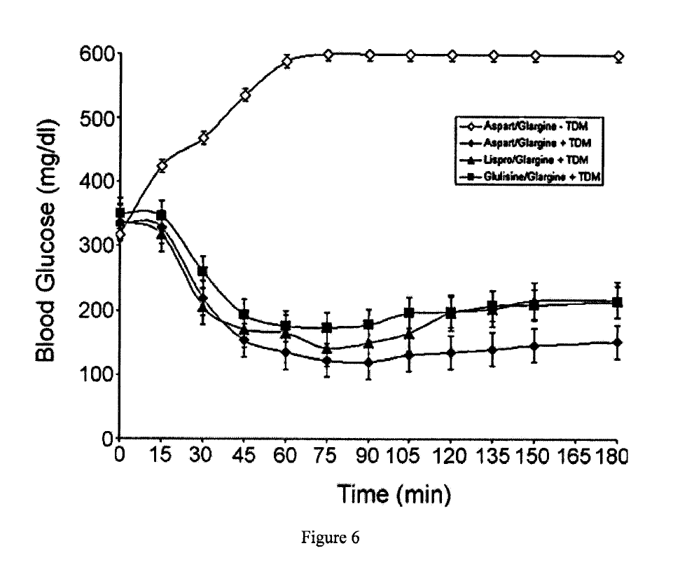

All three formulations were clear and remained clear for several weeks when stored at 4° C. Absorption of insulin was observed with all three mixed insulin formulations that contained TDM, but not with formulations that lacked TDM. All three mixed insulin formulations containing TDM produced rapid insulin absorption from the nasal cavity (Tₘₐₓ=15 minutes), but insulin concentrations remained elevated for more than 180 minutes after administration, a pattern that reflected the combined uptake profiles of fast-acting and long-acting insulins. Of note, the absorption of aspart insulin and lispro insulin was not greater than the absorption of glulisine insulin when each fast-acting analog of insulin was mixed with glargine insulin, a result that was different from the results of experiments depicted in FIG. 1A, where the short acting insulins were tested individually. Importantly, at extended time points after administration of the insulin mixtures (T=60 minutes, 120 minutes and 180 minutes), insulin was present in the plasma at levels far above baseline (>100_U/ml). No such residual insulin was observed in the experimental data presented in FIG. 1A when short acting insulin analogs were tested individually, but it was observed in FIG. 1B when glargine insulin was tested individually. Blood glucose data from these experimental animals are presented in FIG. 6.

FIG. 6 shows blood glucose levels in rats that received 2 Units of a mixed insulin formulation with 0.125% TDM. Each data point represents the mean±SEM of 3-6 animals.

The data are consistent with the insulin absorption results shown in FIG. 5. Animals displayed a rapid fall in blood glucose and a sustained hypoglycemic effect when any of the three mixtures were applied nasally in the presence of TDM.

Discussion: The results described above provide two seminal pieces of information. First, all three forms of fast-acting insulin can be mixed with one long-acting form of insulin, glargine insulin, and formulated with TDM. The other long-acting form of insulin, detemir insulin, could not. Second, all three of the mixed insulin formulations, containing a fast-acting insulin analog and glargine insulin plus TDM, produced a blend of rapid and extended insulin absorption and corresponding hypoglycemic effects when applied nasally. The nasal insulin absorption data obtained in the experiments utilizing mixtures of a fast-acting insulin analog and glargine insulin formulated with TDM are most directly interpreted as follows: the blended response to the insulin mixtures described in FIGS. 5 and 6 include two overlapping and additive events. The initial peak of insulin absorption (Tₘₐₓ=15 minutes) is provided primarily by the absorption of the fast-acting insulin analog. This initial peak of fast-acting insulin absorption is followed by a prolonged period of glargine insulin absorption. This interpretation is consistent with the results obtained when the fast-acting forms of insulin and the long-acting glargine insulin were tested individually (FIGS. 1 and 2).

Previous studies have shown that the concentration of TDM used in these studies, for example 0.125%, provided a robust increase in regular human insulin absorption with minimal toxicity to the cells that line the nasal cavity. At a concentration of 0.25% or 0.50% TDM, slightly more insulin absorption was observed, while at a concentration of 0.06% TDM, incrementally less insulin absorption was observed.

Nasal administration of regular human insulin in the presence of 0.06% and 0.125% TDM resulted in 44% and 55% relative bioavailability respectively when compared to subcutaneous administration of regular insulin. By comparison, nasal administration of regular insulin in the presence of 0.25% and 0.50% TDM resulted in 64% and 77% relative bioavailability when compared to subcutaneous administration of regular insulin. At a concentration of 0.50% TDM, changes in the appearance of the cells that line the nasal cavity became more evident than the minor changes observed at a concentration of 0.125% TDM.

In all of the nasal insulin delivery experiments described in this report, nasal insulin was administered in the form of liquid nose drops, applied from a stock solution by a pipettor. It is anticipated that nasal delivery of insulin formulations using a spray device, to instill small droplets of solution throughout the nasal cavity, will increase the surface area contacted by the insulin and increase the overall amount of insulin absorbed.

The results obtained in cell culture experiments and whole animal experiments are consistent with the hypothesis that the alkylglycoside causes a temporary perturbation of the nasal barrier to drug absorption. Earlier studies have shown that alkylglycosides increase both paracellular transport and transcellular transport of insulin. Substitution of DDM for TDM provided essentially identical results.

While it is possible that an interaction takes place between molecules of the alkylglycoside surfactant and insulin in the nasal formulation prior to administration, direct evidence that this type of interaction directly alters insulin absorption is lacking. Formulations containing 0.125% TDM plus a mixture of glargine insulin with any of the three fast-acting insulins remained clear even after storage for several weeks.

Alkylglycosides such as TDM and DDM have been shown to stabilize protein formulations and increase their effective shelf-life. Hence, in these experiments, TDM may serve a dual role, to both stabilize the mixtures of short-acting and long-acting insulins, and also to serve as an absorption-enhancing agent. This remarkable duality of actions provides an important and unique role for TDM and DDM in the development of an effective stable nasal insulin formulation, whether it contains a single form of insulin or a mixture of fast-acting and long-acting insulins.

While not being bound to any particular theory, the acidic environment of the nasal cavity could certainly present an opportunity for localized foci of glargine insulin precipitation to occur. However, it is not known for certain if long-acting glargine insulin remains in the nose in a depot environment and gradually is released from the depot to be absorbed slowly, as is generally considered to be the case following subcutaneous injections of glargine insulin. The time course of the reversal of TDM action on nasal permeability to insulin becomes very important when considering how, and if, a patient with diabetes mellitus could utilize nasal formulations that contained both fast-acting and long-acting forms of insulin. Optimally, a patient could take a mixed nasal insulin formulation containing TDM at mealtime. Immediate absorption of fact-acting insulin would occur, to provide appropriate coverage for the glycemic burden consumed at the meal. Thereafter, the effect of TDM on the permeability of the nasal cavity would extend for several hours and allow long-acting insulin to be absorbed and provide extended duration of insulin coverage for at least 3 hours after the meal. Additional experimentation will be required to demonstrate that this same scenario holds true in humans and to better define the optimal insulin formulation composition to use (fast-acting and long-acting insulin concentrations and alkylglycoside concentrations can be varied to obtain optimal pharmacodynamic responses).

The rate and extent of absorption of all insulin analogs would be expected to be limited by several factors, including mucociliary clearance of unabsorbed insulin and TDM from the nose, impermeability of the nasal cavity cell surfaces that did not receive an effective dose of TDM because of how and where it was administered, the limited surface area available for absorption, and enzymatic destruction of insulin analogs and TDM, both in the nasal cavity and/or following absorption into the epithelial cells that line the nasal cavity. The distinct chemical composition of the insulin analogs may cause one or more of them to behave differently than the others during one or more of the several different stages of the nasal absorptive process.

It has been reported that mixtures of glargine insulin and either lispro insulin or aspart insulin became cloudy, but were still effective, when injected subcutaneously in humans. No increase in pain or adverse reactions was observed in patients receiving the cloudy insulin mixtures. Detemir insulin was not clear when mixed with 0.125% TDM and it could not be absorbed from the nose into the systemic circulation when mixed with TDM. This form of insulin was the only one of seven insulins tested that was not absorbed from the nose in the presence of TDM. NPH insulin was also cloudy, with or without TDM addition, but NPH insulin was absorbed from the nose when mixed with TDM. In some experiments, animals received nose drops with TDM alone and then the insulins were applied 15 minutes later. Under these conditions, detemir insulin, like aspart insulin and glargine insulin, was absorbed from the nose. Detemir insulin displayed a prolonged duration of action, much like glargine insulin. This type of approach is useful to demonstrate an aspect of TDM action on the nasal cavity, but it is impractical to develop a commercially successful nasal formulation containing detemir insulin if it requires the administration of an alkylglycoside absorption enhancer minutes before a separate application of the insulin. No such requirement exists for a formulation containing glargine insulin.

In summary, three fast-acting insulin analogs (aspart, glulisine, and lispro insulins), were absorbed rapidly following nasal delivery in a formulation containing 0.125% TDM. The long-acting insulin analog, glargine insulin, demonstrated a slower rate of absorption and an extended hypoglycemic effect. Mixtures of long-acting glargine insulin with each of the three different short-acting insulins plus 0.125% TDM were successfully absorbed from the nasal cavity. All three mixtures displayed a pharmacokinetic profile that reflected absorption of both the fast-acting and the long-acting forms of insulin.

Although the invention has been described with reference to the above example, it will be understood that modifications and variations are encompassed within the spirit and scope of the invention. Accordingly, the invention is limited only by the following claims.

What is claimed is:

1. A method of introducing an insulin analog into the circulatory system of a subject comprising administering the insulin analog admixed with an alkylglycoside, thereby introducing the insulin analog to the circulatory system of the subject.

2. The method of claim 1, wherein the insulin analog is a long-acting insulin analog.

3. The method of claim 1, wherein the insulin analog is glargine insulin.

4. The method of claim 2, further comprising administering a fast-acting insulin analog.

5. The method of claim 4, wherein the fast-acting insulin analog is lispro insulin, aspart insulin, glulisine insulin, or combination thereof.

6. The method of claim 3, wherein the AUC₀₋₁₈₀ (μU/ml min) is greater than 60,000 and the Tₘₐₓ (min) is about 60.

7. The method of claim 1, wherein the alkylglycoside has an alkyl chain comprising between 10 to 16 carbons.

8. The method of claim 2, wherein the alkylglycoside is selected from the group consisting of dodecyl maltoside, tridecyl maltoside, tetradecyl maltoside, sucrose mono-dodecanoate, sucrose mono-tridecanoate, and sucrose mono-tetradecanoate.

9. The method of claim 8, wherein the alkylglycoside is tetradecyl-beta-D-maltoside.

10. The method of claim 1, wherein the insulin analog is administered via the oral, buccal, nasal, nasolacrimal, inhalation, pulmonary, transdermal or CSF delivery route.

11. The method of claim 10, wherein the insulin analog is administered via inhalation.

12. A pharmaceutical composition comprising:
a) an insulin analog; and
b) an absorption increasing amount of an alkylglycoside, in a pharmaceutical carrier.

13. The pharmaceutical composition of claim 12, wherein the insulin analog is a long-lasting insulin analog.

14. The pharmaceutical composition of claim 13, wherein the insulin analog is glargine insulin.

15. The pharmaceutical composition of claim 14, further comprising a fast-acting insulin analog.

16. The pharmaceutical composition of claim 15, wherein the fast-acting insulin analog is lispro insulin, aspart insulin, glulisine insulin, or combination thereof.

17. The pharmaceutical composition of claim 12, wherein the alkylglycoside has an alkyl chain including between 10 to 16 carbons.

18. The pharmaceutical composition of claim 17, wherein the alkylglycoside is selected from the group consisting of dodecyl maltoside, tridecyl maltoside, tetradecyl maltoside, sucrose mono-dodecanoate, sucrose mono-tridecanoate, and sucrose mono-tetradecanoate.

19. The pharmaceutical composition of claim 18, wherein the alkylglycoside is tetradecyl-beta-D-maltoside.

20. A method of treating diabetes mellitus in a subject comprising administering to the subject the pharmaceutical composition of claim 12.
